(19) United States
(12) Patent Application Publication
(10) Pub. No.: US 2015/0184120 A1
(43) Pub. Date: Jul. 2, 2015

(54) FOOD WASTE TREATMENT DEVICE USING MICROORGANISMS

(71) Applicant: Waste to Water, LLC, Weatherfield, CT (US)

(72) Inventor: Seuk Hwa Park, Chungju Si (KR)

(21) Appl. No.: 14/580,924

(22) Filed: Dec. 23, 2014

Related U.S. Application Data
(63) Continuation of application No. 12/076,677, filed on Mar. 19, 2008.
Application No. 12/076,677 is a continuation-in-part of application No. 11/725,811, filed on Mar. 20, 2007, now abandoned.

(30) Foreign Application Priority Data
Apr. 7, 2006 (KR) 20060009248

(51) Int. Cl. C12M 1/33; B09B 3/00; B01F 7/00; B01F 7/04; C12M 1/06

(52) U.S. Cl. CPC C12M 45/02; B01F 7/04; C12M 27/06; B01F 7/00583; B09B 3/00

(57) ABSTRACT

The present invention is directed to a composition for decomposing a majority of food waste into water and carbon dioxide comprising an effective combination of at least two species of microorganisms chosen from bacillus, lactobacillus, burkholderia, yeast fungus, eumycetes or any combinations thereof. A preferred embodiment comprises a combination of four different species of microbes having DNA sequences that correspond to SEQ ID Nos. 5-8 and which were deposited with the Korean Collection for Type Cultures (KCTC) on Mar. 8, 2007, and designated KCTC11085BP; KCTC11086BP; KCTC11087BP; KCTC11088BP, respectively. Also, presented by this invention is a device and methods for decomposing a majority of food waste into water and carbon dioxide using the microbial compositions presented herein.

### CROSS-REFERENCE TO RELATED APPLICATIONS

The present application is continuation of U.S. patent application Ser. No. 12/076,677, entitled “FOOD WASTE TREATMENT DEVICE USING MICROORGANISMS” filed Mar. 19, 2008. U.S. patent application Ser. No. 12/076,677 is a continuation-in-part of U.S. Ser. No. 11/725,811, filed Mar. 20, 2007, which claims priority to Korean Utility Model Application No. 2006-0009248, filed Apr. 7, 2006. The contents of U.S. Ser. Nos. 11/725,811, 12/076,677, and Korean Utility Model Application No. 2006-0009248 are hereby incorporated by reference in their entireties.

### SEQUENCE LISTING

The instant application contains a Sequence Listing which has been submitted in ASCII format via diskette and is hereby incorporated by reference in its entirety. Said ASCII copy, filed on Sep. 22, 2008 and accepted on Sep. 24, 2008, can be found on record in U.S. application Ser. No. 12/076,677.

### BACKGROUND OF THE INVENTION

As the food service industry expands, homes and restaurants generate increasingly large amounts of leftover food. Some of the leftover food is used as feed for domestic animals, but due to the difficulty in containing, transporting and/or treating the leftover food, almost all leftover food/food waste is reclaimed or incinerated.

Reclamation methods typically produce foul odors which are generated by nitrogen and sulfur compounds released during the process. In addition, reclamation methods generate a high density leachate that contaminates the atmosphere, water and soil.

Incineration requires incinerators of high capacity and contaminates the atmosphere by releasing harmful substances into the air during incineration. Furthermore, the efficiency of incinerating leftover food is decreased by low-caloric leftover food and also by moisture in leftover food. Therefore, in addition to also releasing dangerous contaminants (like dioxin), this process can also be expensive.

Another method of removing leftover food waste is the dry method system. The dry method system dehydrates food waste by stirring and chopping dried food wastes. However, this method is not practiced by normal households due to the substantial cost of electricity involved.

Yet another method of removing leftover food waste is by decomposition methods. Decomposition methods provide an optimal environment for microorganisms to grow and decompose food wastes into H₂0 and C0₂. These systems sometimes require stirring equipment to mix food wastes in a processing container. In certain situations, such a system may require a separate chopper in order to process tough or hard food wastes. Therefore, this system is not always suitable for small scale operation. In addition, flexible and lengthy materials are often not able to be cut by a blade and can end up being wound around the stirring axis of the container. When food waste becomes wound around a stirring axis, the motor may become overloaded and cause a malfunction or fire.

Additionally, these decomposition methods still yield large amounts of decomposed waste material or sediment. For example, composting may result in only about a 45% decrease in waste material mass—still leaving a significant amount of sediment behind. While the remaining material, or sediment, can be used as compost, such amounts of remaining mass may not be practical for commercial and urban uses.

Thus, there remains a need to develop a useful, economically sound and environmentally safe and effective way to treat leftover food waste.

### SUMMARY OF THE INVENTION

The present invention is directed to a food waste treatment system using a combination of microorganisms that are capable of decomposing the majority of food waste material into water and carbon dioxide, leaving a minimal amount of sediment behind. The microbial mixture provided by this invention is capable of decomposing up to 97% of solid food waste material. In other embodiments, the present invention is directed to a method of treating food waste comprising using a food waste treatment device in conjunction with the combination of microorganisms described herein.

In certain preferred embodiments, the microbial mixture is a combination of two to four species of microbes chosen from bacillus, burkholderia and lactobacillus and combinations thereof.

In a preferred embodiment, the microbial mixture is a combination of four microbes, in the proportion of about 85% having a DNA sequence corresponding to SEQ ID No. 5, about 12% having a DNA sequence corresponding to SEQ ID No. 6, about 1.5% having a DNA sequence corresponding to SEQ ID No. 7 and about 1.5% having a DNA sequence corresponding to SEQ ID No. 8.

### BRIEF DESCRIPTION OF THE DRAWINGS

FIG. 1 depicts the external appearance of an embodiment of the food waste treatment device of the present invention, designated Device A.

FIG. 2 depicts the internal structure of Device A, an embodiment the food waste treatment device of the present invention.

FIG. 3 depicts an example of a stirrer axis and stirrer arms applied to the food waste treatment device of the present invention.

FIG. 4 depicts an example of a bearing installed on a stirring axis applied to the food waste treatment device of the present invention.

FIG. 5 depicts an example of the temperature control and drainage applied to the food waste treatment device of the present invention.

FIG. 6 depicts the 16S rDNA sequence (SEQ ID NO. 1) of a preferred bacteria strain used in the microorganism mixture, designated “B1” in Table 1, as well as a listing of bacterium species having the greatest homology with respect to this partial sequence.

FIG. 7 depicts the 16S rDNA sequence (SEQ ID NO. 2) of a preferred bacteria strain used in the microorganism mixture, designated “B2” in Table 1, as well as a listing of bacterium species having the greatest homology with respect to this partial sequence.

FIG. 8 depicts the 16S rDNA sequence (SEQ ID NO. 3) of a preferred bacteria strain used in the microorganism mixture, designated “B3” in Table 1, as well as a listing of bacterium species having the greatest homology with respect to this partial sequence.

FIG. 9 depicts the 16S rDNA sequence (SEQ ID NO. 4) of a preferred bacteria strain used in the microorganism mixture, designated “B4” in Table 1, as well as a listing of bacterium species having the greatest homology with respect to this partial sequence.

FIG. 10 depicts the rDNA sequence (SEQ ID NO. 5) of a preferred bacteria strain used in the microorganism mixture.

FIG. 11 depicts the rDNA sequence (SEQ ID NO. 6) of a preferred bacteria strain used in the microorganism mixture.

FIG. 12 depicts the rDNA sequence (SEQ ID NO. 7) of a preferred bacteria strain used in the microorganism mixture.

FIG. 13 depicts the rDNA sequence (SEQ ID NO. 8) of a preferred bacteria strain used in the microorganism mixture.

FIG. 14 depicts a production flowchart for the preparation of the microbial mixture for use in the present invention.

FIG. 15 depicts the external appearance of another embodiment of the food waste treatment device of the present invention, designated Device B.

FIG. 16 depicts the right side external appearance of Device B, an embodiment of the food waste treatment device of the present invention.

FIG. 17 depicts the left side external appearance of Device B, an embodiment of the food waste treatment device of the present invention.

FIG. 18 depicts an example of an integrated chopper applied to the food waste treatment device of the present invention.

FIG. 19 depicts the external appearance of the stirrer axis and stirrer arm viewed from the top of FIG. 15.

FIG. 20 depicts the external appearance of an example of a stirrer ann equipped with a shear pin applied to the food waste treatment device of the present invention.

FIG. 21 depicts the external appearance of an example of a stirrer ann equipped with a Poly-Urethane tip applied to the food waste treatment device of the present invention.

FIG. 22 depicts the external appearance of an example of a stirrer axis mounted to a driving axle by using anchor flange method applied to the food waste treatment device of the present invention.

FIG. 23 depicts the external appearance of a filler material called “BIO-HELPER” applied to the food waste treatment device of the present invention.

FIG. 24 depicts the microscopic appearance of the filler material called “BIO-HELPER” applied to the food waste treatment device of the present invention.

FIG. 25 depicts the filler material called “BIO-HELPER” applied to the food waste treatment device of the present invention.

### DETAILED DESCRIPTION OF THE PREFERRED EMBODIMENTS

The present invention is directed to a food waste treatment composition used, with or without a device also described by this invention, which utilizes a particular combination of microbes to decompose the majority of food waste material and convert that food waste into water, carbon dioxide and a small amount of sediment.

The microbial mixture of the present invention is prepared and mixed with the food waste material. Using the appropriate microbial mixture results in decomposition of most of the food waste into water and carbon dioxide. In some embodiments of the microbial mixture, greater than 55% of the solid food waste is decomposed. In a preferred embodiment, up to 97% of the solid food waste is decomposed.

In certain embodiments, the microbial mixture comprises from 1 to about 10 different strains of bacteria. In certain preferred embodiments, the microbial mixture comprises about two to four different strains of microbial species. In yet other preferred embodiments, the mixture comprises four (4) different stains of microbial species.

Because the microbial mixture of this invention is capable of decomposing about 97% of the food waste material, many of the issues associated with other methods of food waste removal and/or treatment are alleviated. By reducing up to 97% of the solid food waste, the problems associated with large amounts of solid waste remaining (as with, e.g., composting) is eliminated. Furthermore, with the generation of mostly water as a by-product, much of the foul odor associated with decomposing organic waste is eliminated, as is the noxious leachates that can result from other decomposition methods. Additionally, the presently described compositions and methods are not costly and eliminate that inefficiencies associated with incineration, dehydration or other mechanical means of eliminating organic waste.

### The Microbial Mixture:

The microbes used in the compositions of the present invention may include species of bacillus, lactobacillus, burkholderia, actimomyces, yeast fungus, eumycetes, as well as combinations thereof. The mixture is capable of decomposing the majority of food waste materials, including protein, starch, grease and cellulose; thus, one or more of the microbes of the mixture must be capable of decomposing long chain structures which are inherent in fats and starches of food waste.

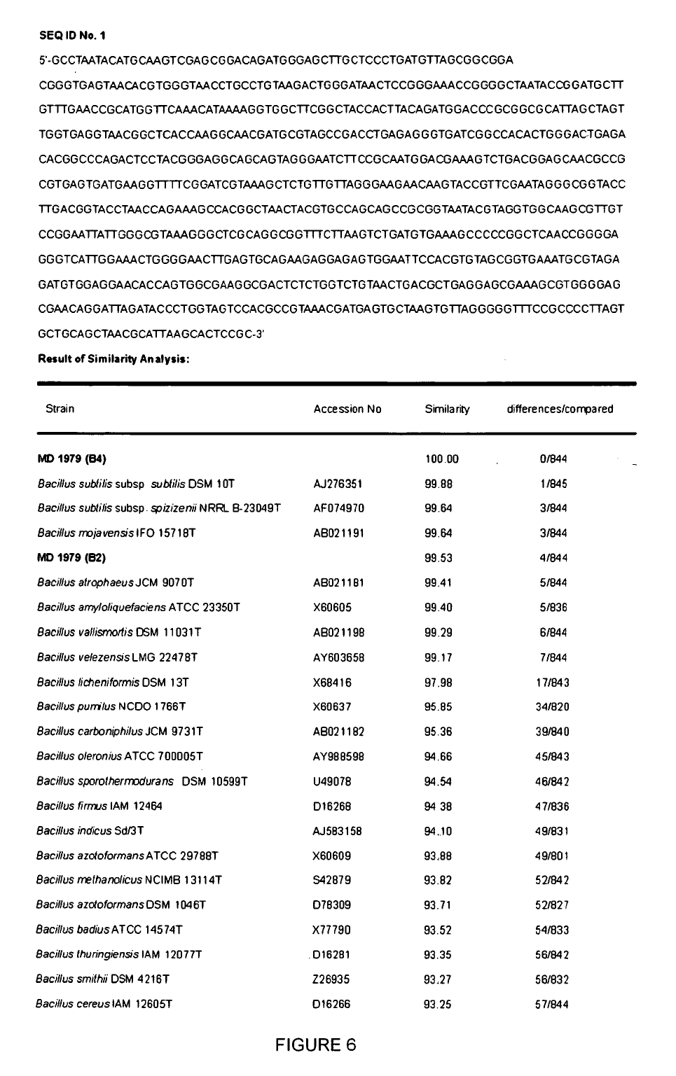

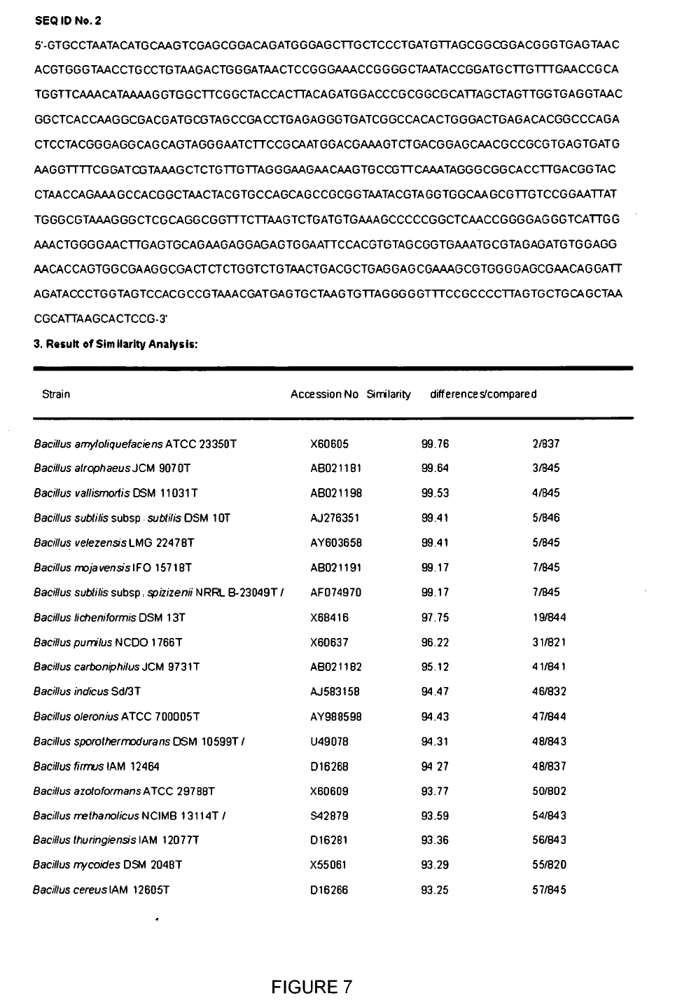

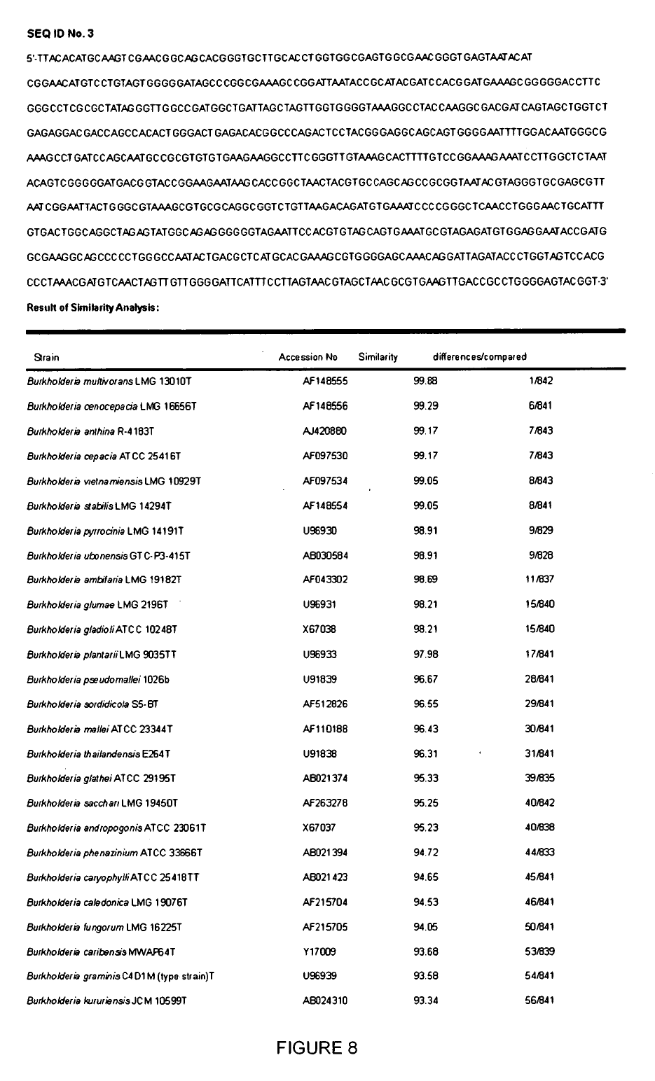

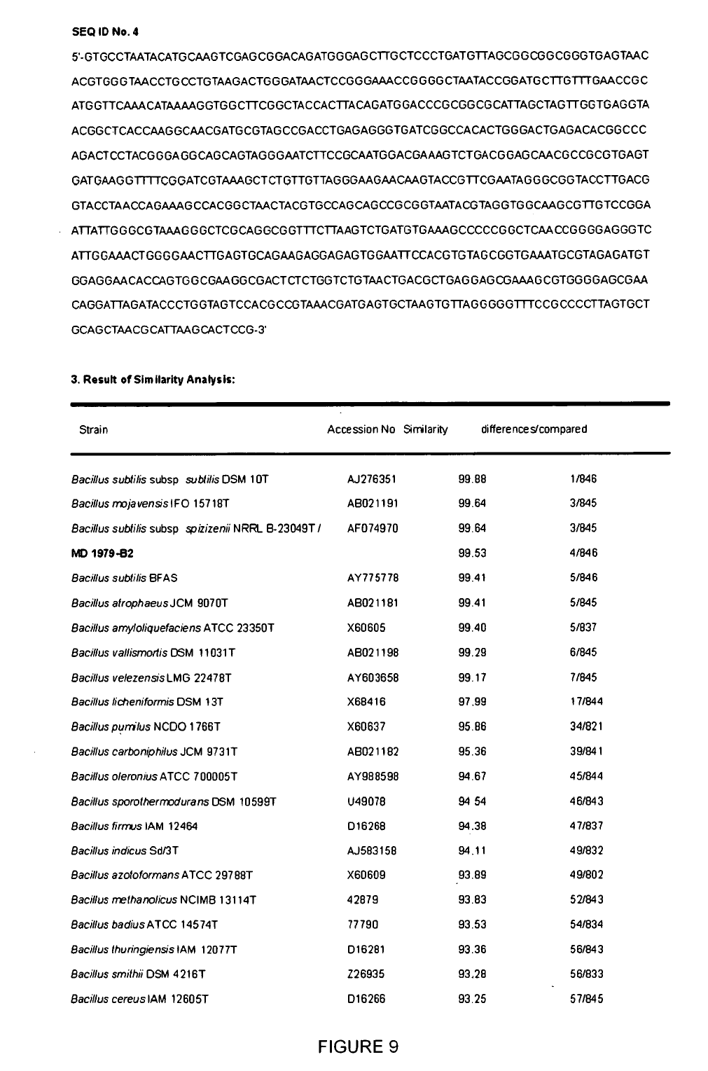

The choice of microbes to add to the mixture and the proportion in which they are added may depend upon the general chemical composition of the food waste intended to be decomposed. The particular mixture of microbes used in a mix may be altered to more efficiently decompose waste of a particular composition. So, for example, a mixture may contain more or less of a given species of microbe to accommodate waste that is known to have a greater protein, fat or carbohydrate content. Thus, by way of an example, a preferred embodiment of this invention comprises four strains of bacteria, designated “B1”, “B2”, “B3” and “B4”. Their partial (16S rDNA) DNA sequences are provided in FIGS. 6-9 and are designated as SEQ ID Nos. 1-4, respectively. In this exemplary embodiment, “B1” was chosen as the predominant strain because of its ability to decompose carbon-nitrogen series of organic matter, as well as its ability to decompose some fat. “B2” was added for its ability to decompose starches and fats which have very long chain molecular structures. The overall time for decomposition of food waste is shortened by the addition of “B2” and its ability to break the long-chain structures of food wastes like starches and fats.

Additionally, when choosing microbes for the mixture, stability, vitality, adaptability, safety and resolvability of the microbes is also taken into consideration. In light of these criteria, strains such as streptomyces sp., cellulosinicrobium funkei, brucella sp., arthrobacter sp. and paenibacillus cookie may also be useful in the microbial mixture.

The mixture of the invention may contain anywhere from 1 to 10 strains of bacillus, lactobacillus, burkholderia, actimomyces, yeast fungus, eumycetes, as well as combinations thereof.

In a more preferred embodiment, the microbial composition is a mixture of three different bacillus species and one lactobacillus species. In a most preferred embodiment, the three bacillus species have DNA sequences corresponding to SEQ ID No. 5, SEQ ID No. 6 and SEQ ID NO. 8 and the lactobacillus species has a DNA sequences corresponding to SEQ ID No. 7.

In certain other preferred embodiments, the invention is a mixture of three different bacillus strains and one burkholderia strain, with: about 85% of a bacillus subtilis species, designated as “B1” in Table 1; about 12% of a second bacillus subtilis species, designated as “B2” in Table 1; about 1.5% of a third bacillus subtilis species, designated as “B4” in Table 1; and about 1.5% of a burkholderia species, designated as “B3” in Table 1:

TABLE 1
A Preferred Embodiment of Microbial Mixture

| | % | | % Similarity |
|---|---|---|---|
| | Composition | Closest Known Strains Based Partial Sequence | to Closest |
| Identifier | in Mixture | (16S rDNA Sequence) | Strains |
| B1 | 85% | MD 1979 (B4) | 100.00 |
| (SEQ ID | | Bacillus subtilis subsp. subtilis DSM 10T | 99.88 |
| No. 1) | | Bacillus subtilis subsp. spizizenii NRRL B-23049T | 99.64 |
| | | Bacillus mojavensis IFO 15718T | 99.64 |
| | | MD 1979 (B2) | 99.53 |
| | | Bacillus atrophaeus JCM 9070T | 99.41 |
| | | Bacillus amyloliquefaciens ATCC 23350T | 99.40 |
| | | Bacillus vallismortis DSM 11031T | 99.29 |
| | | Bacillus velezensis LMG 22478T | 99.17 |
| | | Bacillus licheniformis DSM 13T | 97.98 |
| | | Bacillus pumilus NCDO 1766T | 95.85 |
| | | Bacillus carboniphilus JCM 9731T | 95.36 |
| | | Bacillus oleronius ATCC 700005T | 94.66 |
| | | Bacillus sporothermodurans DSM 10599T | 94.54 |
| | | Bacillus firmus IAM 12464 | 94.38 |
| | | Bacillus indicus Sd/3T | 94.10 |
| | | Bacillus azotoformans ATCC 29788T | 93.88 |
| | | Bacillus methanolicus NCIMB 13114T | 93.82 |
| | | Bacillus azotoformans DSM 1046T | 93.71 |
| | | Bacillus badius ATCC 14574T | 93.52 |
| | | Bacillus thuringiensis IAM 12077T | 93.35 |
| | | Bacillus smithii DSM 4216T | 93.27 |
| | | Bacillus cereus IAM 12605T | 93.25 |
| B2 | 12% | Bacillus amyloliquefaciens ATCC 23350T | 99.76 |
| (SEQ ID | | Bacillus atrophaeus JCM 9070T | 99.64 |
| No. 2) | | Bacillus vallismortis DSM 11031T | 99.53 |
| | | Bacillus subtilis subsp. subtilis DSM 1OT | 99.41 |
| | | Bacillus velezensis LMG 22478T | 99.41 |
| | | Bacillus mojavensis IFO 15718T | 99.17 |
| | | Bacillus subtilis subsp. spizizenii NRRL B-23049T | 99.17 |
| | | Bacillus licheniformis DSM 13T | 97.75 |
| | | Bacillus pumilus NCDO 1766T | 96.22 |
| | | Bacillus carboniphilus JCM 9731T | 95.12 |
| | | Bacillus indicus Sd/3T | 94.47 |
| | | Bacillus oleronius ATCC 700005T | 94.43 |
| | | Bacillus sporothermodurans DSM 10599T | 94.31 |
| | | Bacillus firmus IAM 12464 | 94.27 |
| | | Bacillus azotoformans ATCC 29788T | 93.77 |
| | | Bacillus methanolicus NCIMB 13114T | 93.59 |
| | | Bacillus thuringiensis IAM 12077T | 93.36 |
| | | Bacillus mycoides DSM 2048T | 93.29 |
| | | Bacillus cereus IAM 12605T | 93.25 |
| B3 | 1.5% | Burkholderia multivorans LMG 13010T | 99.88 |
| (SEQ ID | | Burkholderia cenocepacia LMG 16656T | 99.29 |
| No. 3) | | Burkholderia anthina R-4183T | 99.17 |
| | | Burkholderia cepacia ATCC 25416T | 99.17 |
| | | Burkholderia vietnamiensis LMG 10929T | 99.05 |
| | | Burkholderia stabilis LMG 14294T | 99.05 |
| | | Burkholderia pyrrocinia LMG 14191T | 98.91 |
| | | Burkholderia ubonensis GTC-P3-415T | 98.91 |
| | | Burkholderia ambifaria LMG 19182T | 98.69 |
| | | Burkholderia glumae LMG 2196T | 98.21 |
| | | Burkholderia gladioli ATCC 10248T | 98.21 |
| | | Burkholderia plantarii LMG 9035TT | 97.98 |
| | | Burkholderia pseudomallei 1026b | 96.67 |
| | | Burkholderia sordidicola S5-BT | 96.55 |
| | | Burkholderia mallei ATCC 23344T | 96.43 |
| | | Burkholderia thailandensis E264T | 96.31 |
| | | Burkholderia glathei ATCC 29195T | 95.33 |
| | | Burkholderia sacchari LMG 19450T | 95.25 |
| | | Burkholderia andropogonis ATCC 23061T | 95.23 |
| | | Burkholderia phenazinium ATCC 33666T | 94.72 |
| | | Burkholderia caryophylli ATCC 25418TT | 94.65 |
| | | Burkholderia caledonica LMG 19076T | 94.53 |
| | | Burkholderia fungorum LMG 16225T | 94.05 |
| | | Burkholderia caribensis MWAP64T | 93.68 |
| | | Burkholderia graminis C4D1M (type strain)T | 93.58 |
| | | Burkholderia kururiensis JCM 10599T | 93.34 |
| B4 | 1.5% | Bacillus subtilis subsp. subtilis DSM 10T | 99.88 |
| (Seq ID | | Bacillus mojavensis IFO 15718T | 99.64 |
| No. 4) | | Bacillus subtilis subsp. spizizenii NRRL B-23049T | 99.64 |
| | | MD 1979-B2 | 99.53 |
| | | Bacillus subtilis BFAS | 99.41 |
| | | Bacillus atrophaeus JCM 9070T | 99.41 |
| | | Bacillus amyloliquefaciens ATCC 23350T | 99.40 |
| | | Bacillus vallismortis DSM 11031T | 99.29 |
| | | Bacillus velezensis LMG 22478T | 99.17 |
| | | Bacillus licheniformis DSM 13T | 97.99 |
| | | Bacillus pumilus NCDO 1766T | 95.86 |
| | | Bacillus carboniphilus JCM 9731T | 95.36 |
| | | Bacillus oleronius ATCC 700005T | 94.67 |
| | | Bacillus sporothermodurans DSM 10599T | 94.54 |
| | | Bacillus firmus IAM 12464 | 94.38 |
| | | Bacillus indicus Sd/3T | 94.11 |
| | | Bacillus azotoformans ATCC 29788T | 93.89 |
| | | Bacillus methanolicus NCIMB 13114T | 93.83 |
| | | Bacillus badius ATCC 14574T | 93.53 |
| | | Bacillus thuringiensis IAM 12077T | 93.36 |
| | | Bacillus smithii DSM 4216T | 93.28 |
| | | Bacillus cereus IAM 12605T | 93.25 |

Note:
While B1 and B4 demonstrate the 16S rDNA sequence, they appear differently shaped under a microscope.

In a most preferred embodiment, the microbial mixture comprises about 85% of a bacillus species the DNA sequence of SEQ ID NO. 5; about 12% of a second bacillus species the DNA sequence of SEQ ID NO. 6; about 1.5% of a third bacillus species having the DNA sequence of SEQ ID NO. 8; and about 1.5% of a lactobacillus species having the DNA sequence of SEQ ID NO. 7. In this embodiment, the bacillus species having the DNA sequence of SEQ ID No. 5 was chosen as the predominant strain because of its ability to decompose carbon-nitrogen series of organic matter, as well as its ability to decompose some fat. The bacteria having the DNA sequence of SEQ ID No. 6 was added for its ability to decompose starches and fats which have very long chain molecular structures. The overall time for decomposition of food waste is shortened by the addition of the bacteria with SEQ ID No. 6 and its ability to break the long-chain structures of food wastes like starches and fats. This microbial mixture (being a combination of microbes having the DNA sequences shown in SEQ ID Nos. 5-8) was deposited with the Korean Collection for Type Cultures (KCTC) on Mar. 8, 2007; these four species were designated KCTC11085BP (SEQ ID No. 5), KCTC11086BP (SEQ ID No. 6), KCTC11087BP (SEQ ID No. 7) and KCTC11088BP (SEQ ID No. 8), by the KCTC.

The bacteria strains used in the present invention may be artificially made or naturally occurring.

Preferred species of bacillus include amyloliquefaciens, subtilis, subtilis subsp. subtilis, subtilis subsp. spizizenii, mojavensis, atrophaeus, vallismortis, velezensis, licheniformis, pumilus, carboniphilus, oleronius, sporothermodurans, firmus, indicus, azotoformans, methano/icus, badius, thuringiensis, smithii, cereus, mycoides and combinations thereof.

Preferred species of lactobacillus include, for example, lactobacillus acidophilus, lactobacillus plantarum, lactobacillus brevis, lactobacillus sakei subsp. sakei, lactobacillus brevis, lactobacillus delbrueckii subsp. bulgaricus, lactobacillus casei, lactobacillus delbrueckii, lactobacillus fermentum, lactobacillus helveticus, lactobacillus plantarum, lactobacillus reuteri, lactobacillus sanfranciscensis and combinations thereof.

Preferred strains of burkholderia include multivorans, cenocepacia, anthina, vietnamiensis, stabilis, pyrrocinia, ubonensis, ambifaria, glumae, gladioli, plantarii, pseudomallei, sordidicola, mallei, thailandensis, glathei, sacchari, andropogonis, phenazinium, caryophylli, caledonica, fungorum, caribensis, graminis, kuruiensis and combinations thereof.

Optionally, the microbial mixture is added to a “filler” material. This filler, sometimes also referred to as a “moisture controller” in this disclosure, provides an environment in which the microbes can thrive and reproduce. The filler material of the present invention may be husks (such as chaff or rice hulls), wood chips or synthetic materials such as PolyEster. Ina preferred embodiment, cedar wood chips are used as the filler.

When using a filler, the filler is soaked in the microbial mixture. The filler serves the additional purpose of maintaining the original concentration of microbes added to a food mixture. The microbes reside in, or on, the solid filler material and therefore, decrease the risk of microbes being “flushed” out with the water by-product of the invention.

When using filler, up to 40% of a container volume of a food waste treatment device (an example of such a device is described hereinbelow) may be consumed by the microbial soaked filler. The filler material needs minimal replacement in the device and provides space for microorganisms to thrive and reproduce. For example, when treating approximately 100 lbs of food waste per day, the microbial mixture was found to be effective for approximately 1 year—after which the microorganisms must be re-inoculated. The used filler material, when using husks or wood chips, may be used as fertilizer.

A very small amount of yeast fungus and eumycetes reside naturally in some filler material, e.g., wood chips. However, in a bacillus-predominant environment of some of the preferred embodiments of the microbial mixture, the eumycetes are unable to produce spores, and therefore comprise less than about 1% of the microbial mixture.

In preferred embodiments, the present invention provides a composition for decomposing a majority of food waste into water and carbon dioxide comprising an effective combination of at least two species of microorganisms chosen from bacillus, lactobacillus, burkholderia, yeast fungus, eumycetes or any combinations thereof. In more preferred embodiments, at least one of the strains of microorganisms is capable of decomposing long chain starches. In other preferred embodiments, the combination of microorganisms comprises at least one species of bacillus and at least one species of lactobacillus, and in more preferred embodiments, the combination of microorganisms comprises four species of microorganisms. In most preferred embodiments, each of the four species of microorganisms has a DNA sequence of SEQ ID No. 5, SEQ. ID No. 6, SEQ. ID No. 7 and SEQ. ID No. 8, and may comprise at least 85% SEQ ID No. 5, about 12% SEQ ID No, about 1.5% SEQ ID No. 7 and about 1.5% SEQ ID No. 8.

In preferred embodiments, the composition further comprises a filler material. In more preferred embodiments, the filler material is a husk, a wood chip (M., cedar wood chips), a man-made filler or combinations thereof.

In preferred embodiments, the composition as described herein, decomposes greater than 55% of the food waste, and in most preferred embodiments, about 97% of the food waste is decomposed.

In certain embodiments, the present invention provides a method for decomposing greater than about 55% of food waste material into water and carbon dioxide comprising: (i) adding an effective amount of a combination of at least two species of microorganisms chosen from bacillus, lactobacillus, burkholderia, yeast fungus, eumycetes and any combinations thereof; and (ii) mixing the food waste material and the combination of microorganisms for a time sufficient to allow the combination of microorganisms to decompose a majority of the food waste into water and carbon dioxide.

In preferred embodiments, greater than about 97% of the food waste material is decomposed by the above method.

In preferred embodiments, the combination of microorganisms used in the above method comprises at least one species of bacillus and at least one species of lactobacillus. In preferred embodiments, at least one of the microorganisms is capable of decomposing long chain starches. In more preferred embodiments, the combination of microorganisms comprises four species of microorganisms, and in even more preferred embodiments, the four species of microorganisms has a DNA sequence of SEQ ID No. 5, SEQ ID No. 6, SEQ ID No. 7, and SEQ ID No. 8, and may comprise at least 85% having SEQ ID No. 5, about 12% SEQ ID No. 6, about 1.5% having SEQ ID No. 7 and about 1.5% having SEQ ID No. 8.

In preferred embodiments, the above method further comprises soaking a filler material with the elective combination of microorganisms. In more preferred embodiments, the filler material is a husk, a wood chip (e.g., a cedar wood chip), a man-made filler or combinations thereof.

In preferred embodiments, 1 gram of the elective combination of microorganisms is mixed with about 110 kilograms of food waste material.

In certain embodiments of the present invention, the composition for decomposing a majority of food waste into water and carbon dioxide comprises an effective combination of at least two Bacillus species selected from the group consisting amyloliquefaciens subtilis, subtilis subsp. subtilis, subtilis subsp. spizizenii, mojavensis, atrophaeus, vallismortis, velezensis, licheniformis, pumilus, carboniphilus, oleronius, sporothermodurans, firmus, indicus, azotoformans, methanolicus, badius, thuringiensis, smithii, cereus, mycoides and combinations thereof. In certain embodiments, the composition comprises an effective combination of at least two bacterial species selected from the group consisting of Bacillus amyloliguefaciens, Bacillus mojavensis and Bacillus pumilus. In certain other embodiments, the composition further comprises a species of Pseudomonas or Burkholderia. Preferably, the species of Pseudomonas is Pseudomonas fluorescens. Examples of the Burkholderia species are set forth in Table 1. In certain embodiments of the present invention, the composition for decomposing a majority of food waste into water and carbon dioxide comprises an effective combination of Bacillus amyloliguefaciens, Bacillus mojavensis, Bacillus pumilus, and Pseudomonas fluorescens. Preferably, the Bacillus amyloliguefaciens is the Bacillus amyloliguefaciens strain having the ATCC No. 23842. Preferably, the Bacillus mojavensis is the Bacillus mojavensis strain having the ATCC No. 51516. Preferably, the Bacillus pumilus is the Bacillus pumilus strain having the ATCC No. 14884. Preferably, the Pseudomonas fluorescens is the Pseudomonas fluorescens strain having the ATCC No. 13525. In certain embodiments, the majority of food waste decomposed is greater than 55%, preferably at least 90%, and yet more preferably about 97%, of the food waste.

In certain embodiments of the present invention, the CFUs of each of the bacterial species present in the composition constitutes at least about 10%, preferably at least 25% of all CFUs in the composition. Thus, for example, when the composition comprises Bacillus amyloliguefaciens, Bacillus mojavensis, Bacillus pumilus and Pseudomonas fluorescens, the CFUs of each of said bacterial species constitutes at least about 10%, preferably at least about 25%, of the total CFUs of the four species in the composition.

In certain embodiments of the present invention, the bacterial compositions are mixed with a filler material. The filler material may be husks (such as chaff or rice hulls), wood chips or synthetic materials, such as polypropylene. Preferably, cedar wood chips are used as the filler. More preferably, the filler material comprises BIOHELPER.

Preferably, the filler material comprises a porous structure of polypropylene, also referred to herein as “BIO-HELPER.” In certain embodiments, BIO-HELPER comprises polypropylene, foaming agent, absorbent and silicon. The present invention also includes a method of preparing BIO-HELPER and the BIO-HELPER prepared by said method, wherein the method comprises the steps of (i) mixing polypropylene with additives to prepare a mixed base formula; (ii) liquefying said base formula, preferably in an airtight container; (iii) extruding said liquid through a mold; and (iv) cooling down said liquid. The resulting product may be cut into polypropylene pellets, for example, pellets 2 mm wide and 2 mm long. The additives may comprise a foaming agent, an absorbent and/or water. In one embodiment, about 88% polypropylene, about 5% foaming agent, about 3% absorbent, about 0.20% water and about 3% silicon, by weight, are mixed to prepare the base formula. Preferably, the polypropylene filler is highly porous, for example, one with the BET Surface Area of about 0.07 or greater, when tested using KS L ISO 18757.

In certain embodiments, when the bacterial composition is present in a powder form, it is mixed with a substrate, such as corn cob fragments or wood chips, to form a product, also referred to as a “decomposition product.” The decomposition product is preferably mixed with the filler material and put into a food waste treatment device before the decomposition process. Once the microbes are inoculated into the main device and become active (e.g., by temperature, moisture and food), they reposition themselves from the substrate into the filler material.

In certain embodiments, the decomposition product contains at least about 7×10⁹ CFUs, preferably at least about 2.0×10¹⁰ CFUs, of the bacterial species per gram of the decomposition product. In certain embodiments, when the bacterial composition is in a powder form, an adherent may be added in mixing said composition with the substrate to allow the bacteria to adhere to said substrate, preferably without causing the substrate material to adhere to each other. For example, glycerine or molasses may be used as an adherent when the bacterial mixture is mixed with com-cob fragments or wood chips.

When the decomposition product contains at least about 7×10⁹ CFUs, preferably at least about 2.0×10¹⁰ CFUs, of the bacterial species per gram of the decomposition product, at least about 2 pounds of the decomposition product may be used, for example, for decomposing about 250 pounds of food waste; at least about 3 pounds of the product for about 500 pounds of the food waste; at least about 4 pounds of the product for about 1000 pounds of the food waste, about 5 pounds of the product for about 1500 pounds of the food waste.

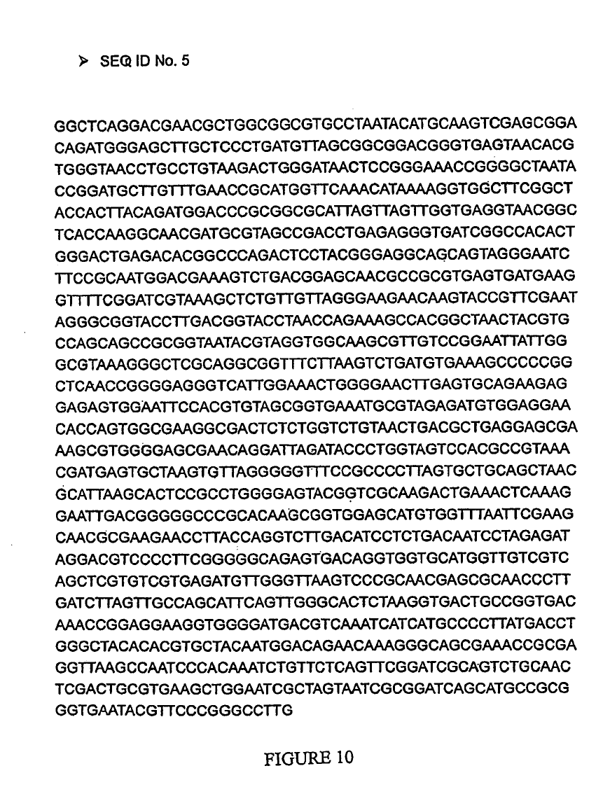

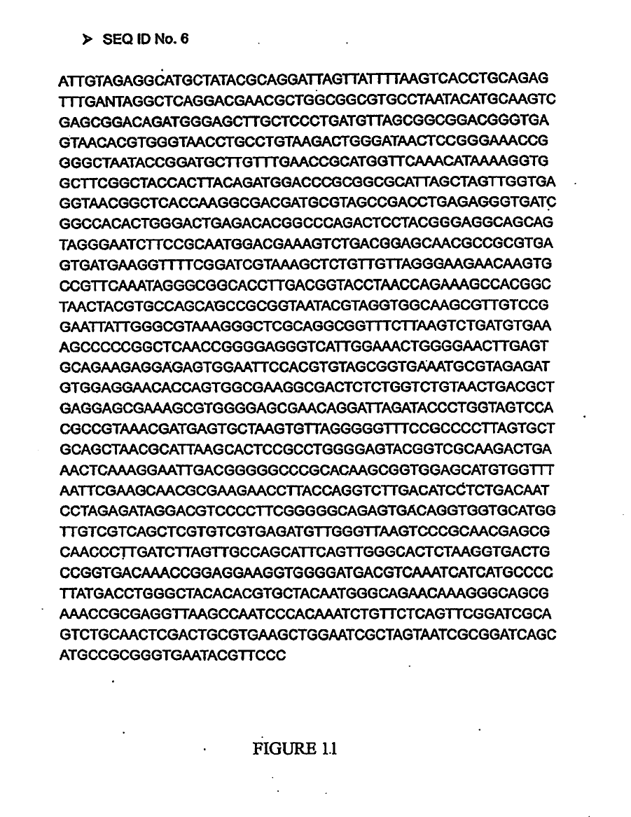

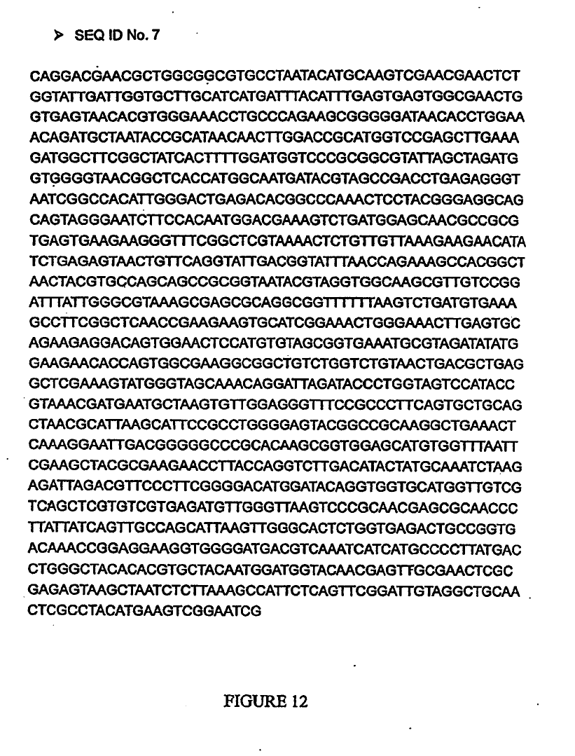

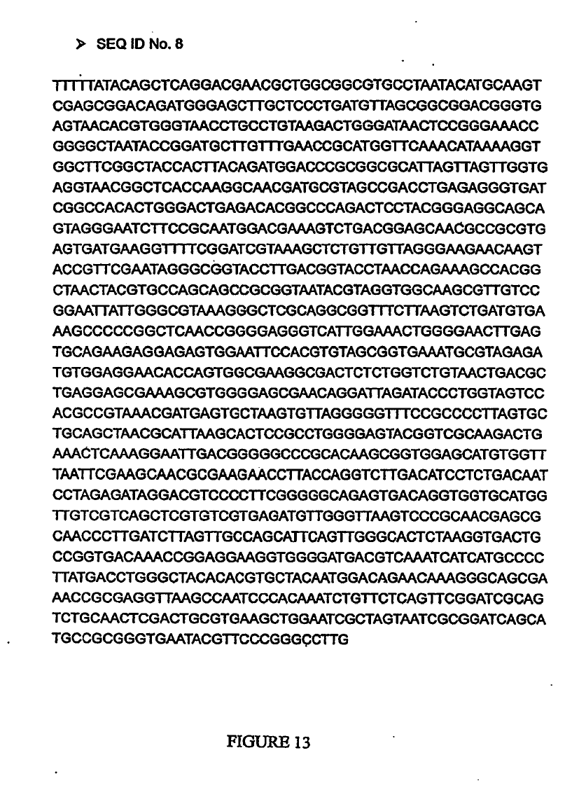

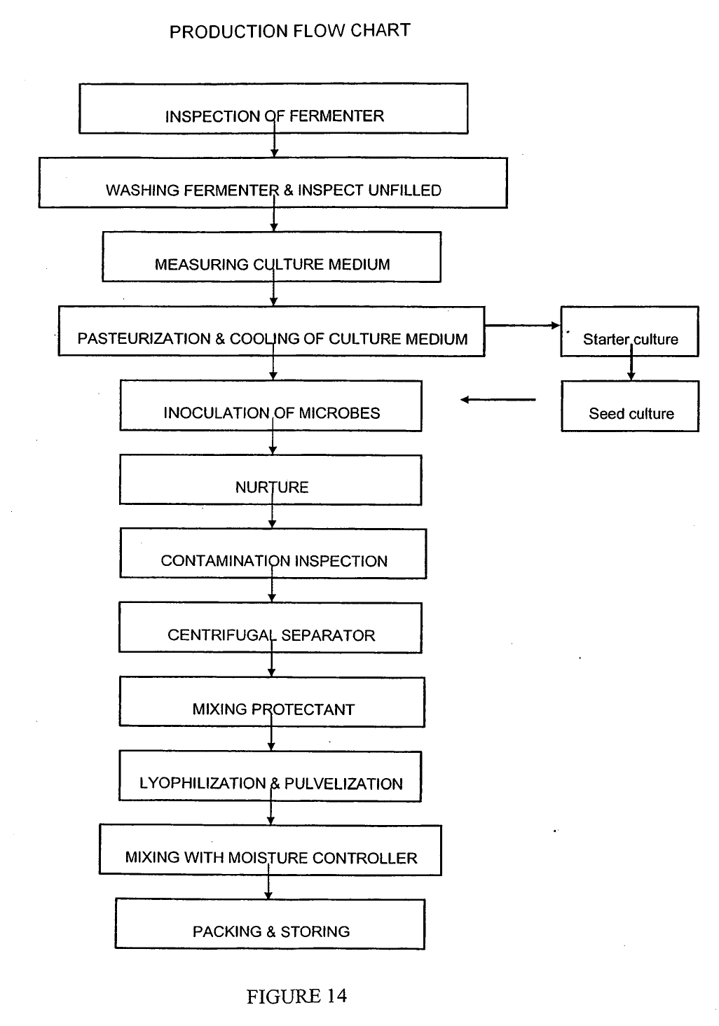

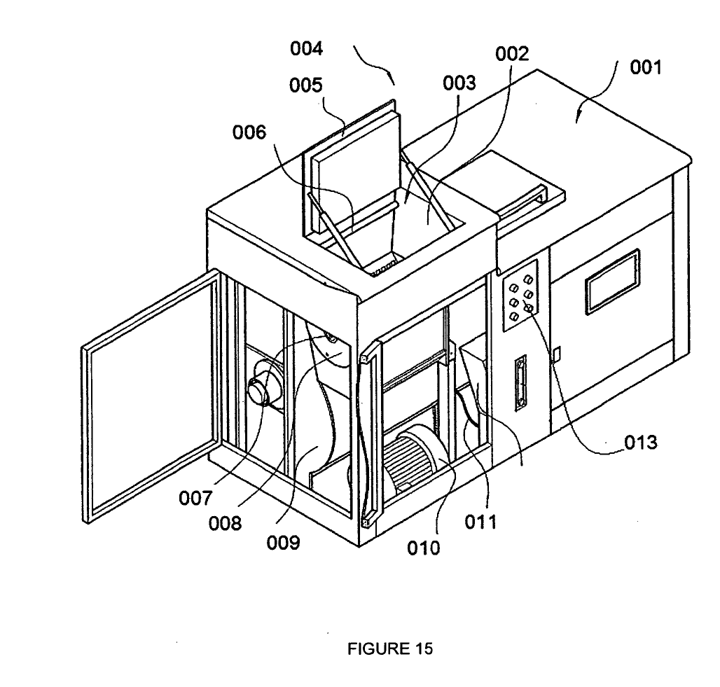

Also provided by this invention is a method of making the microbial mixture. Procedures for generating and preserving microbial mixtures are known in the art. Preferably, the microbial mixture of the present invention is formulated through lyophilization, which maximizes the number and the vitality of the microbes. FIG. 10 is a flow chart showing the procedures used to make the microbial mixture of the present invention; a detailed description is also provided in Example 1 hereinbelow. During lyophilization and pulverization, a protectant may be used. The protectant may be any known pasteurized composition, such as maltodextrin, trehalose, glucose or skim milk. This list is not exhaustive, as one of skill in the art would recognize other pasteurized compositions which would be useful in the present invention.

### The Food Waste Treatment Device:

The food waste treatment device of the present invention may be any device capable providing maximum contact of the microbial mixture with the food waste and a drainage opening to filter out the water which results from the decomposition of food waste.

In certain preferred embodiments, the present invention is directed to a device for decomposing a majority of food waste material into water and carbon dioxide comprising: a processing container for receiving food waste material, having sides, a bottom and a lid and containing a composition for decomposing a majority of food waste material into water and carbon dioxide comprising an effective combination of at least two species of microorganisms chosen from bacillus, lactobacillus, burkholderia, yeast fungus, eumycetes or any combinations thereof; a stirring axis having a first end and a second end, the first end being attached to a side of the processing container and the second end being attached to an opposite side of the processing container; at least one stirrer arm attached to the stirring axis; a filter screen located near the bottom of the processing container; and a water discharge outlet connected to the processing container and located opposite the filter screen.

In certain embodiments of the present invention, the composition for decomposing a majority of food waste into water and carbon dioxide comprises an effective combination of at least two Bacillus species selected from the group consisting amyloliquefaciens, subtilis, subtilis subsp. subtilis, subtilis subsp. spizizenii, mojavensis, atrophaeus, vallismortis, velezensis, licheniformis, pumilus, carboniphilus, oleronius, sporothermodurans, firmus, indicus, azotoformans, methanolicus, badius, thuringiensis, smithii, cereus, mycoides and combinations thereof. In certain embodiments, the composition comprises an effective combination of at least two bacterial species selected from the group consisting of Bacillus amyloliguefaciens, Bacillus mojavensis and Bacillus pumilus. In certain other embodiments, the composition further comprises a species of Pseudomonas or Burkholderia. Preferably, the species of Pseudomonas is Pseudomonas fluorescens. Examples of the Burkholderia species are set forth in Table 1. In certain embodiments of the present invention, the composition for decomposing a majority of food waste into water and carbon dioxide comprises an effective combination of Bacillus amyloliguefaciens, Bacillus mojavensis, Bacillus pumilus, and Pseudomonas fluorescens. Preferably, the Bacillus amyloliguefaciens is the Bacillus amyloliguefaciens strain having the ATCC No. 23842. Preferably, the Bacillus mojavensis is the Bacillus mojavensis strain having the ATCC No. 51516. Preferably, the Bacillus pumilus is the Bacillus pumilus strain having the ATCC No. 14884. Preferably, the Pseudomonas fluorescens is the Pseudomonas fluorescens strain having the ATCC No. 13525. In certain embodiments, the majority of food waste decomposed is greater than 55%, preferably at least 90%, and yet more preferably about 97%, of the food waste.

In certain embodiments of the present invention, the stirring axis of the device is connected to a driving axel for rotating the stirring axis in a manner that allows the stirring axis to be easily replaced without removing any remaining food wastes in the processing container. Preferably the stirring axis is mounted on the driving axel by using an anchor flange. In certain embodiments, the stirring axis further comprises a blade.

In certain embodiments of the present invention, the device comprises a plurality of stirrer arms, one end of each of said stirrer arms being attached to the exterior of the stirring axis in a way that allows said stirrer arm to become a single of point of failure if the stir arm gets jammed during the decomposing process. Preferably, each of the stirrer arms is attached to the stirring axis by a shear pin. Disposed at the other end of each of the stirrer arms is a blade. Preferably, the blade comprises a polyurethane tip that is designed to fracture if subjected to a heavy force, to protect the stirrer arm from breakage.

In certain embodiments of the present invention, the device further comprises an integrated chopper for reducing the size of the food waste before it is put in the processing container. Preferably, the integrated chopper comprises a chopper axel rotated by a chopper motor, a plurality of spiral shaped blades mounted on the chopper axel in series; and a guide for guiding a turning radius of the chopping blades, said guide comprising a plurality of teeth extending toward the chopper axel, and a plurality of gaps separating the extended teeth, said gaps providing a plurality of chopper channels through which the blades pass as they are rotated by the chopper motor. Preferably the gaps are about the same width as the chopper channels, and the blades chop the food waste as they pass through the chopper channels.

In more preferred embodiments, the device further comprises a control panel attached to the processing container for controlling the speed of the stirring axis, temperature, and humidity inside the processing container. In more preferred embodiments, the humidity in the processing container of the device is maintained at about 65% to about 75% and the temperature in the processing container is maintained at about 20° C. to about 35° C. In even more preferred embodiments, the control panel further controls oxygen supply to the processing container.

In preferred embodiments, the stirrer arm of the device is bladed. In other preferred embodiments, at least one of the stirrer arms is “U” shaped. In more preferred embodiments, at least one of the stirrer arms is located at the outer surface of a stirring axis. In certain preferred embodiment the stirrer arm is arranged in at least a 90 degree angle. In other preferred embodiments, the center of at least one stirrer arm consists of a sharp cutter. In other preferred embodiments, either side of the stirring axis comprises a blade which is located across the stirring axis.

In preferred embodiments of the present invention, the device further comprises bearings located on both side of the stirring axis. In preferred embodiments, the bearings comprise a cap and a bushing. The cap is mounted on the inner wall of the processing container and the bushing is inserted at the inner center of the cap to pierce through the stirring axis. In certain embodiments, the bearings are made of non-metallic material to prevent corrosion and rust due to salinity contained by food waste.

In preferred embodiments, there is drainage that stores decomposed food wastes on the lower part of discharge outlet. In such an embodiment, one end of drainage may be connected with sewerage and the other end may be connected with a water supply to wash off remaining food wastes. In more preferred embodiments, sufficient pitch is provided to the side that is connected with sewerage.

In certain preferred embodiments, at least one of the species of microorganisms placed into the processing container is capable of decomposing long chain starches. In certain preferred embodiments, one of the species of microorganisms is eumycetes. In other preferred embodiments, the combination of microorganisms in the device comprises at least one species of bacillus and at least one species of lactobacillus. In more preferred embodiments, the combination of microorganisms comprises four species of microorganisms. In even more preferred embodiments, each of the four species of microorganisms has a DNA sequence of SEQ ID No. 5, SEQ. ID No. 6, SEQ. ID No. 7 and SEQ. ID No. 8, and may comprise at least 85% SEQ ID No. 5, about 12% SEQ ID No. 6, about 1.5% SEQ ID No. 7, and about 1.5% SEQ ID No. 8.

In certain embodiments, the processing container of the device further contains a filler material. In preferred embodiments, the filler material is a husk, a wood chip (e.g., cedar wood chips), a man-made filler or combinations thereof, and in most preferred embodiments, the filler material occupies up to about 40% of the volume of the processing container.

In preferred embodiments, the device is capable of decomposing greater than about 55% of the food waste, and in even more preferred embodiments, the device is capable of decomposing greater than about 97% of the food waste.

In certain preferred embodiments, the present invention provides a food waste treatment device, also referred to as “an advanced device,” which comprises a process container for receiving food waste material and a composition for decomposing a majority of the food waste material into water and carbon dioxide; a stirrer axis (also referred to as “a stirring axis”) rotatably attached to the sides of the processing container, and a driving axel for rotating the stirrer axis, wherein the stirrer axis is amounted on the driving axel by an anchor flange; and a plurality of stirrer arms, one end of each of said stirrer arms being attached to the exterior of the stirrer axis by a shear pin and the other end comprising a blade. In certain embodiments, the blade of the stirrer arm comprises a polyurethane blade. In certain embodiments, the stirrer axis further comprises a blade. Preferably, the device comprises at least 4, more preferably at least 7 stirrer arms.

In certain embodiments of the present invention, the advanced device further comprises an integrated chopper for chopping the food waste before it is received by the processing container. Preferably, the integrated chopper comprises a chopper axel, a plurality of spiral shaped blades mounted on the chopper axel in series, and a guide for guiding a turning radius of the chopping blades, said guide comprising a plurality of teeth extending toward the chopper axel and a plurality of gaps separating the extended teeth. The gaps providing a plurality of chopper channels through which the blades pass as they are rotated by the chopper motor. Preferably, the gaps of the extended teeth are of substantially the same width as the chopper channels.

In certain embodiments of the present invention, the advanced device further comprises a filter screen located near the bottom of the processing container and a water discharge outlet connected to the processing container and located opposite the filter screen.

In certain embodiments of the present invention, the advanced device further comprises a control panel for controlling the rotation speed of the stirring axis, temperature and humidity of the processing container. Preferably, the humidity in the processing container of the advanced device is maintained at about 65% to about 75% and the temperature in the processing container is maintained at about 20° C. to about 35° C. Preferably, the control panel further controls oxygen supply to the processing container.

In certain preferred embodiments, at least one of the species of microorganisms placed into the processing container of the advance device is capable of decomposing long chain starches. In certain preferred embodiments, one of the species of microorganisms is eumycetes. In other preferred embodiments, the combination of microorganisms in the device comprises at least one species of bacillus and at least one species of lactobacillus. In more preferred embodiments, the combination of microorganisms comprises four species of microorganisms. In even more preferred embodiments, each of the four species of microorganisms has a DNA sequence of SEQ ID No. 5, SEQ. ID No. 6, SEQ. ID No. 7 and SEQ. ID No. 8, and may comprise at least 85% SEQ ID No. 5, about 12% SEQ ID No. 6, about 1.5% SEQ ID No. 7, and about 1.5% SEQ ID No. 8.

In certain embodiments of the present invention, the composition for decomposing a majority of food waste into water and carbon dioxide comprises an effective combination of at least two Bacillus species selected from the group consisting amyloliquefaciens, subtilis, subtilis subsp. subtilis, subtilis subsp. spizizenii, mojavensis, atrophaeus, vallismortis, velezensis, licheniformis, pumilus, carboniphilus, oleronius, sporothermodurans, firmus, indicus, azotoformans, methanolicus, badius, thuringiensis, smithii, cereus, mycoides and combinations thereof. In certain embodiments, the composition comprises an effective combination of at least two bacterial species selected from the group consisting of Bacillus amyloliguefaciens, Bacillus mojavensis and Bacillus pumilus. In certain other embodiments, the composition further comprises a species of Pseudomonas or Burkholderia. Preferably, the species of Pseudomonas is Pseudomonas fluorescens. Examples of the Burkholderia species are set forth in Table 1. In certain embodiments of the present invention, the composition for decomposing a majority of food waste into water and carbon dioxide comprises an effective combination of Bacillus amyloliguefaciens, Bacillus mojavensis, Bacillus pumilus, and Pseudomonas fluorescens. Preferably, the Bacillus amyloliguefaciens is the Bacillus amyloliguefaciens strain having the ATCC No. 23842. Preferably, the Bacillus mojavensis is the Bacillus mojavensis strain having the ATCC No. 51516. Preferably, the Bacillus pumilus is the Bacillus pumilus strain having the ATCC No. 14884. Preferably, the Pseudomonas fluorescens is the Pseudomonas fluorescens strain having the ATCC No. 13525. In certain embodiments, the majority of food waste decomposed is greater than 55%, preferably at least 90%, and yet more preferably about 97%, of the food waste.

In certain embodiments, the processing container of the advanced device further contains a filler material. In preferred embodiments, the filler material is a husk, a wood chip, (e.g., cedar wood chips), a man-made filler or combinations thereof, and in most preferred embodiments, the filler material occupies up to about 40% of the volume of the processing container.

In preferred embodiments, the advanced device is capable of decomposing greater than about 55% of the food waste, and in even more preferred embodiments, the device is capable of decomposing greater than about 97% of the food waste.

### An Embodiment of the Food Waste Treatment Device of the Present Invention: Device A

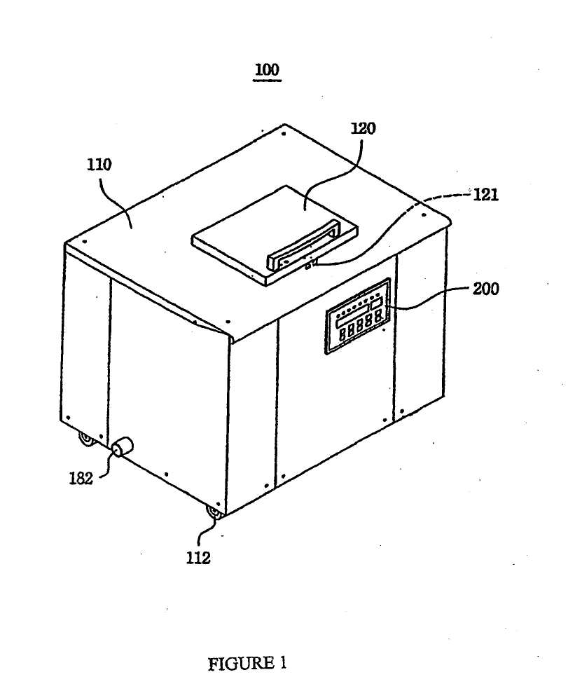

One embodiment of the food waste treatment device of the present invention, designated Device A, is depicted in FIGS. 1-5. Device A provides optimal results when utilized with the microbial mixture of the present invention.

The figures in the drawings show the subject matter of the invention highly schematically and should be understood as not being to scale. The individual components of the subject matter according to the invention are represented so that their structure can be clearly shown.

FIG. 1 shows the main body (110) of the food waste treatment device which contains the processing container (130) which decomposes the food waste with microorganisms. The cover (120) is located at upper part of the main body. Hinge joints are used for cover (120). The main body (110) has wheels (112) attached to the bottom for ease of mobility. The opening of main body has a sensor (121) so the machine only operates when cover is completely closed. The control panel (200) is located in the front of the main body (110).

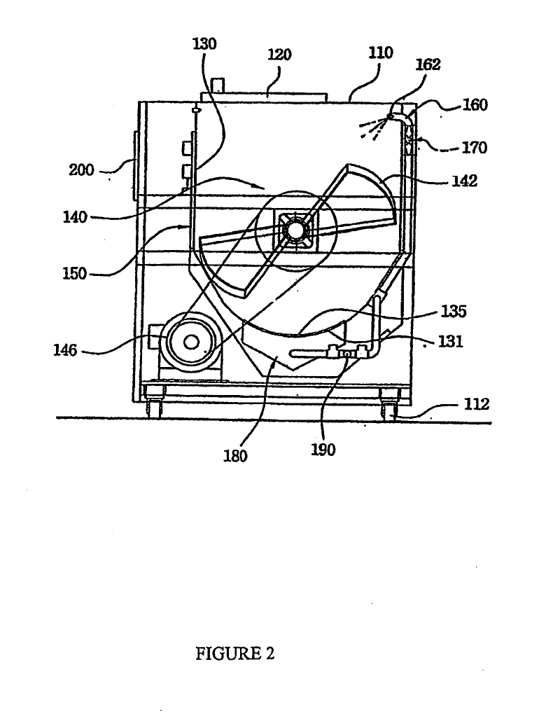

FIG. 2 shows the internal structure of the food waste treatment device. A filtering screen (135) is placed at the bottom of the processing container (131). In order to maximize the efficiency, it is recommended to use two or three stirrer rums (142). A driving motor (146) rotates the axis of the stirrer rums (142).

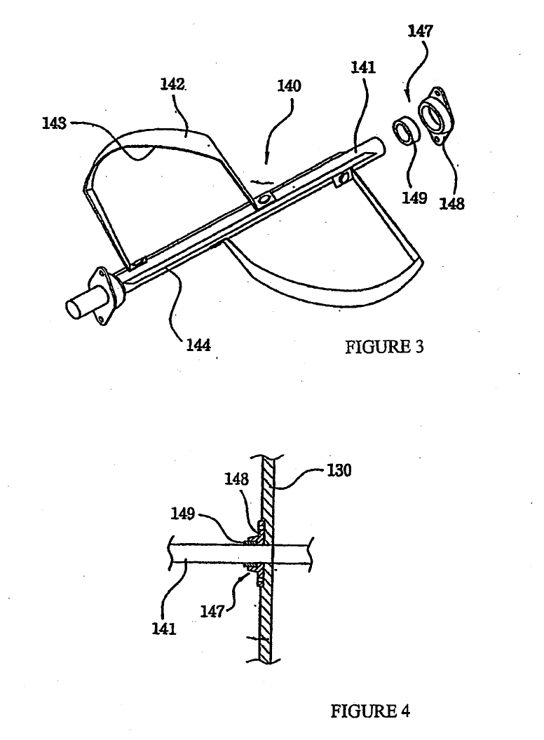

FIG. 3 shows the processing container (130). There are one or more “U” shape stirrer rums (142) on the outer surface of the stirring axis (141). In order to maximize the efficiency, it is recommended to use two or three stirrer rums (142). The stirrer (140) is located crossways within the processing container (130). One end of the stirrer (140) is fixed to the side of processing container (130) and has a driving motor to rotate the stirring axis (141). One end of stirrer rum (142) is fixed to the stirring axis (141) and the other end of the stirrer rum (142) is fixed to the center of the stirrer axis (141). At this point, it is recommended to arrange each rum at least 90 degrees tilted. The stirrer rum and outer surface of stirring axis have sharp blades attached (143 and 144, respectively).

Blades of the stirrer rum (143) break the food waste that is in the processing container (130) and the axis blades (144) break the food waste that is entangled in the stirring axis (141)

A small amount of food waste may not be broken down, however, the pressure from the heavy load pushes the waste the food is then broken down by the axis blades (144).

FIG. 4 shows the bearings (147) installed on the stirring axis (141). Both ends of the stirring axis (141) are installed to the side of processing container (131) to allow for rotation. One end of stirring axis (141) penetrates processing container (130) and connects with the motor which is located on the outer surface of the processing container (130).

Both ends of stirrer axis (141) have bearings (147) for easier rotation of axis. Bearings that are used for the axis are preferably made of non-metallic material to prevent corrosion and rust due to salinity contained by food waste.

Furthermore, bearings (147) used at the each end of the stirring axis (141) consist of a cap (148) that is mounted on the inner wall of processing container (130) and a bushing (149), which is inserted at the inner center of the cap (148) to penetrate the stirring axis (141). A Packing gasket is placed to prevent moisture from food waste.

A temperature control (150) is installed at the outer wall of the processing container (130). Thin plates of heating element units (152) are placed for temperature control. The heating element units (152) are wrapped by insulation (154), and it surrounds the processing container (130). The processing container (130) has its own temperature sensor to keep the certain temperature.

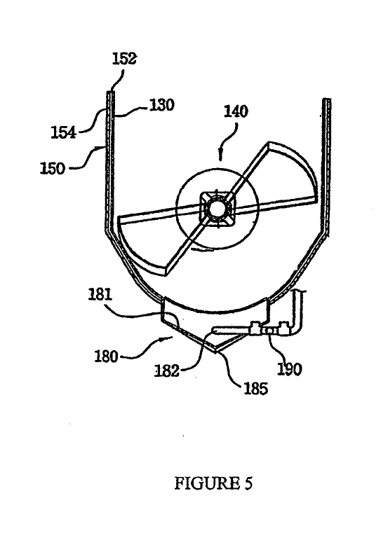

FIG. 5 depicts the temperature control and drainage of the food waste treatment device. The moisture control (160) is installed at the upper part of processing container (130). It consists of nozzles (162) to check the moisture level of processing unit.

A water discharge is installed to wash off food waste from processing container that was filter through the filter screen (135). The water discharge consists of a discharge outlet (180) a drainage (181) is located at the lower part of discharge outlet (180). One end of drainage is connected with sewerage. One end of sewerage is connecting to the solenoid valve (190) which has water spray nozzles (182) for drainage (181).

The solenoid valve (190) supplies water to the nozzles (182), moisture control (160) and water discharge (180). This device may restrain the activities of microorganisms because of lack of oxygen supply if the cover is closed. Therefore, oxygen supply is one of the most important elements to maximize the activities of the microorganisms. A ventilation fan, also referred to as an oxygen supplier (170), is located on top of the inner walls of processing container (130).

The control panel (200) controls the main body (110), stirrer (140), temperature control (150), moisture control (160), oxygen supplier (170) and water discharge (180) with appropriate settings for time and temperature. In preferred embodiments, the humidity of the processing container is maintained at about 65% to about 75%, and the temperature of the processing container is maintained at about 20° C. to about 35° C.

To give further understanding of this device, the stirrer axis (141) rotates when the power is on (Timer: Approx. 5 min.) Then water is sprayed into the processing container (Solenoid valve timer: Approx. 30 Min.), which is controlled by moisture control (160). Broken down organisms will filter through the filter screen (135) and water discharge (180). Water spray nozzles will wash off drainage and transfer the broken organisms to sewage. After the process of the water spray nozzles are completed, the stirrer will stop itself. The process of break down will begin by microorganisms. When all of the processes are completed, the system will repeat itself by returning to the first step.

### An Embodiment of the Food Waste Treatment Device of the Present Invention: Device B

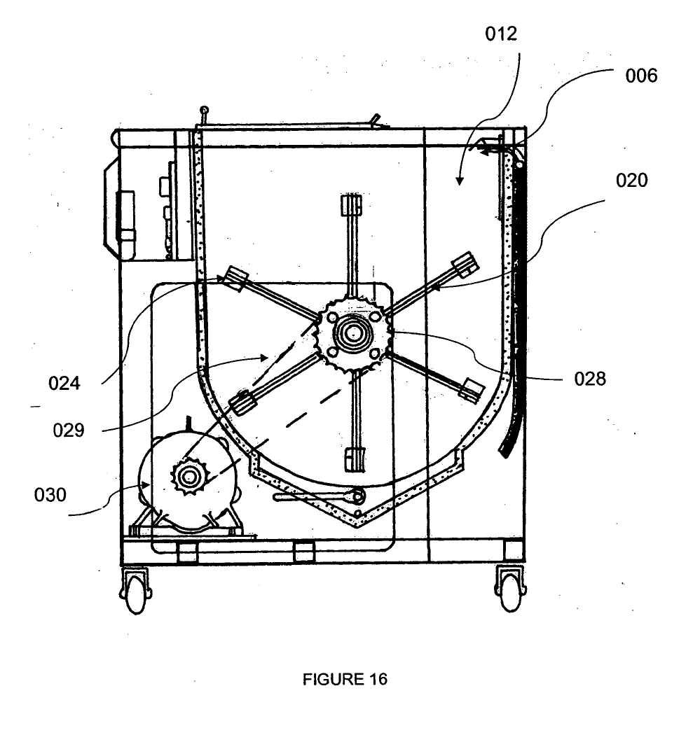

Another embodiment of the food waste treatment device of the present invention, designated Device B, is depicted in FIGS. 15-24. Device B is an updated version of Device A and an example of an advanced food waste treatment device of the present invention. Device B contains an integrated chopper that allows the reduction of the size of the food waste before it is put into the processing container. In addition, the designs of the stirrer axis and stirrer arms have been modified from those of Device A.

The figures in the drawings, once again, show the subject matter of the invention highly schematically and should be understood as not being to scale. The individual components of the subject matter according to the invention are represented so that their structure can be clearly shown.

FIG. 15 shows the main body (001) of Device B, an embodiment of the food waste treatment device of the present invention, which contains an integrated chopper (003) for reducing the size of the food waste before it is being subjected to the decomposition process using microorganisms (??). Preferably, the integrated chopper (003) chops food wastes into not more than 2 inches and automatically feed the chopped food wastes into a processing container (012) for the next process. This chopper can be operated under either manual or auto mode. In order to switch between manual and auto mode, as a safety feature, a designated key is provided to operate the control panel (013). In addition, under the auto mode, the top door (005) must be kept closed to begin the operation.

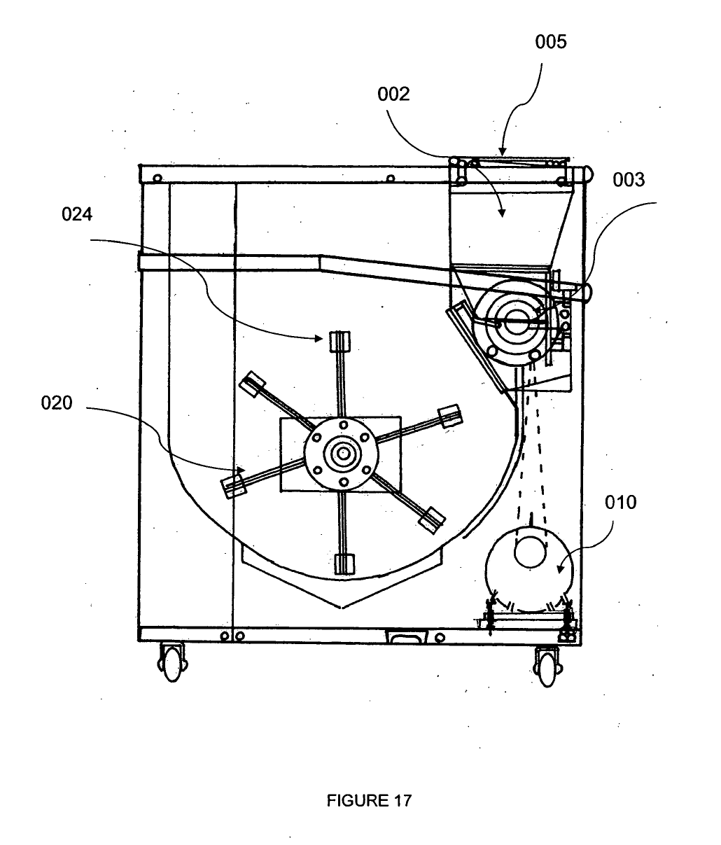

FIG. 16 shows the right side view of Device B, and FIG. 17 shows the left side view the device.

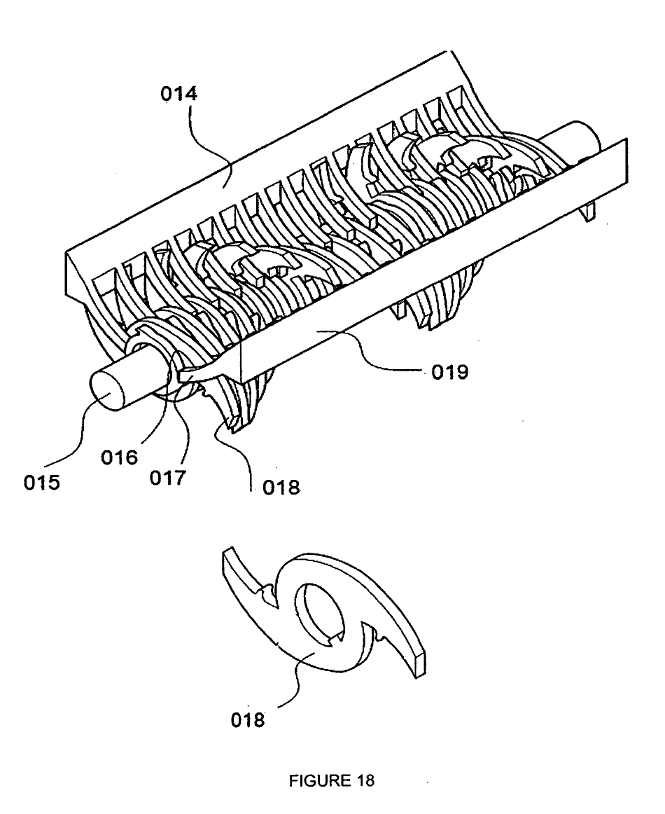

FIG. 18 shows the chopper (003) that is integrated to Device B. Located inside a hopper (002), the chopper includes a chopper axle (007) that rotates by chopper motor (010), plurality of chopping blades (018) that are mounted in series on the chopper axle (007), and a guide (019) that has a plurality of chopper channels (016) which guide a turning radius of chopping blades (018) and chops food waste with the chopping blades (018). The guide (019) is constituted by a number of extended teeth (017), like a comb, towards to the chopper axle (015), with the gaps between the extended teeth providing the chopper channels (016). The width of the chopper channels (016) is preferably the same as the thickness of the chopping blades (018). Food waste is chopped as the chopping blades (018) pass through the chopper channels (016). As indicated in FIG. 18, the chopping blades (018) are mounted in spiral shape on the chopper axle (007), which maximized the chopping ability by having chopping blades working one after the other. The hopper (002) is designed as funnel shape and has a door (005) on top. The hopper (002) is also equipped with electronic sensor which located in between the top door (005) and surface of the chopper (003), where the top door (005) contacts. When the top door (005) opens, the chopper (003) stops and when the top door (005) closes, the chopper (003) starts again automatically. A water spray nozzle (006) is installed on the top of the hopper (002) to wash off remains on the chopping blades (018), the chopper axle (007) and the inside of the hopper (002).

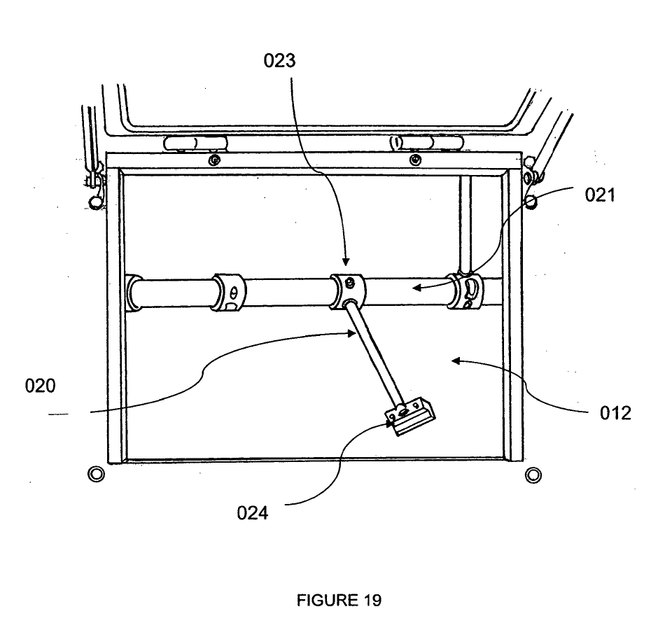

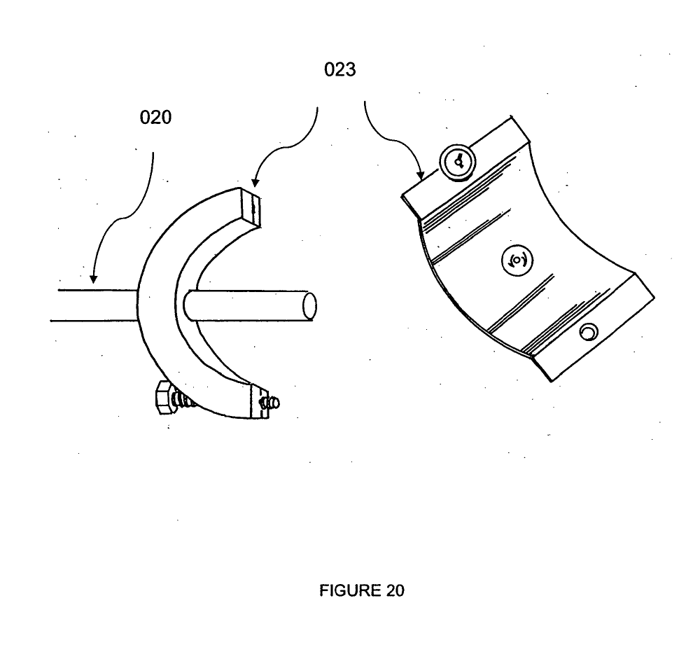

FIG. 19 shows the inside of the processing container (012). FIG. 20 shows the external appearance of a stirrer arm (020) equipped with a shear pin (023) applied to the food waste decomposer device of the present invention. The shear pin mechanism works as a single point of failure should the stirrer arm (020) become jammed by an object, such as a piece of metal inadvertently be dropped into the stirrer drum. This shear pin is designed to fail if subjected to a heavy force to protect the drive sprockets (028), chain (029), motor (030) and stirrer axis (021) from severely damaged.

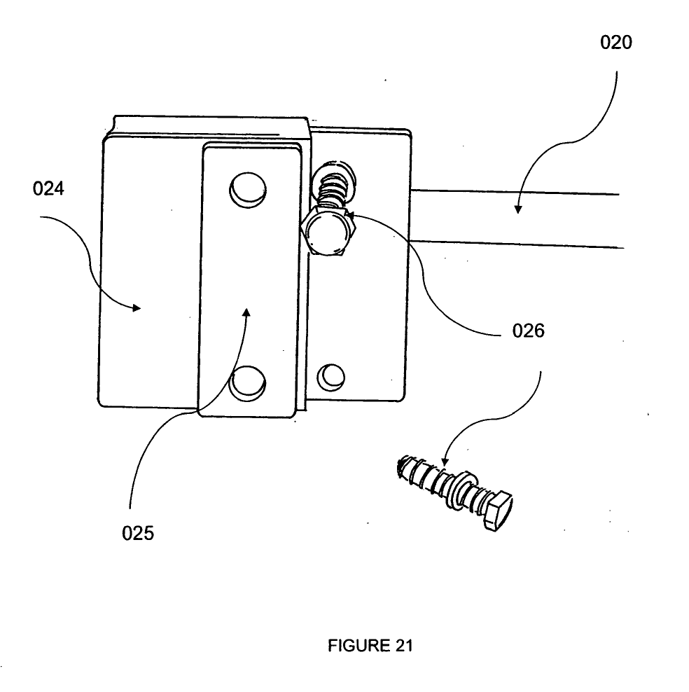

FIG. 21 shows the external appearance of the stirrer axis (021) equipped with a Poly-Urethane tip (024) applied to the food waste treatment device of the present invention. This Poly-Urethane tip (024) is designed to fracture if subjected to a heavy force to protect the stirrer arm (021) from breakage. Once the Poly-Urethane tip has fractured, the tip can be easily replaced by unscrewing a backing plate (025) by removing blade bolts (026).

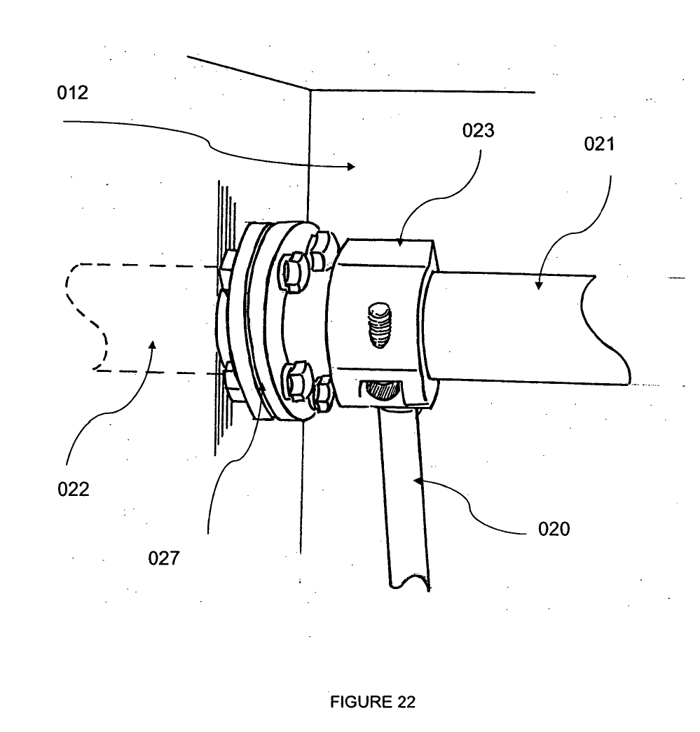

FIG. 22 shows the external appearance of the stirrer axis (021) mounted to a driving axle (022) by using anchor flange (027) method applied to the food waste treatment device of the present invention. This anchor flange (027) mount method is design to replace stirrer axis (021) easily without removing any remaining food wastes in the processing container (012).

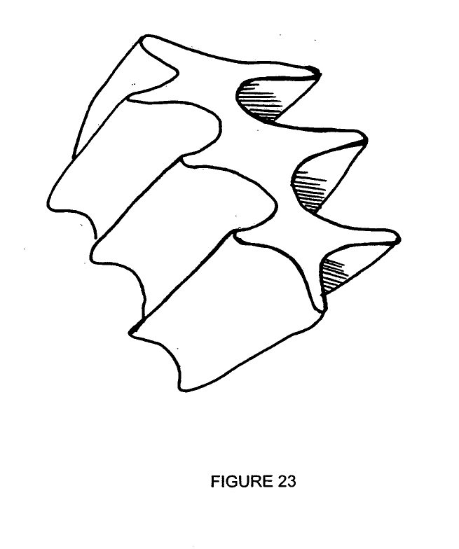

FIG. 23 depicts the external appearance of a filler material called “BIO-HELPER” applied to the food waste treatment device of the present invention. This filler, sometimes also referred to as a “moisture controller” in this disclosure, provides an environment in which the microbes can thrives and reproduce. The filler material of the present invention, Poly-Easter synthetic material is used. This filler is designed to offer a maximum living surface and contains numerous amount of moisture for microbes to thrive and reproduce. (Question: what is this BIO-HELPER?? Would any polyester material work or is there a special formula??)

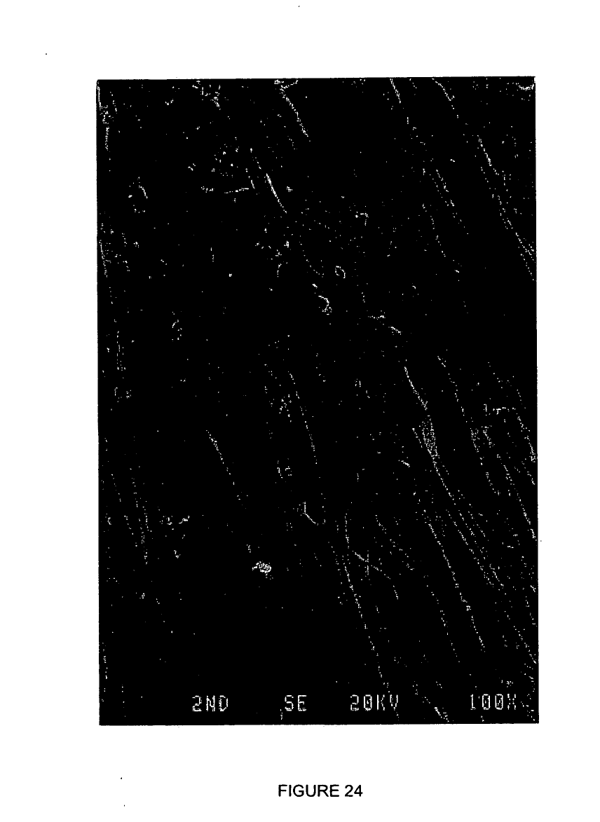

FIG. 24 depicts the microscopic appearance of the filler material called “BIO-HELPER” to the food waste decomposer device of the present invention.

Device B offers several advantages as a food waste treatment device. By having a chopper integrated into the main body of the device, the food waste can be broken down into smaller pieces before it is subjected to the decomposition process, which increases the decomposing capacity, reduces processing time and conserves electricity. The device is also equipped with the stirrer arms with a built-in shear pin mechanism as a single point of failure should the stirrer arm become jammed by an object, such as a piece of metal inadvertently dropped into the processing container. This shear pin is designed to fail if subjected to a heavy force to protect the drive sprockets, chain, motor and stirrer axis from being severely damaged. In addition, the stirrer arms are equipped with polyurethane blades, which are designed to fracture if subjected to a heavy force to protect the stirrer arm from breakage. And by having an anchor flange mounted stirrer axis, the device allows the stirrer axis to be replaced easily without removing any remaining wastes in the processing container.

### EXAMPLES

### Example 1

### Preparation of Microbial Mixture

Microbes used: Bacillus subtilis (3 kinds) and Lactobacillus

TABLE 2
Culture Medium

| RAW MATERIAL | % |
|---|---|
| Peptone | 2 |
| Yeast Extract | 1 |
| Glucose | 2 |
| Sodium Acetate | 0.1 |
| Ammonium citrate | 0.1 |
| Sodium carbonate | 0.05 |
| K₂HPO₄ | 0.1 |
| MgSO₄ | 0.01 |
| MnSO₄ | 0.005 |
| ZnSO₄ | 0.001 |

1% of each spawn was inoculated to the culture medium. The culture medium, set forth in Table 2 (above), was added in the order of peptone, yeast extract and glucose to the culture fluid and dissolved completely at 140° F. Culture medium was pasteurized for at least 15 minutes at about 250° F. at 1.2 hPa, with no negative air pressure, and a pressure higher than 0.5 vvm of positive air when cooling after pasteurization was complete, maintaining constant water volume throughout the process. The volume of water used was the same before and after pasteurization of the culture medium.

Microbes were cultivated via depths nurture method (air supply and stirring) for 18-30 hours at 77° F.-98.6° F. and a pH of between about 6.0 and 7.0.

After cultivation process, microbes were collected by standard collection procedures, such as in a continuance centrifugal separator.

### Lyophilization Method:

The collected body of microbes was mixed with an appropriate portion of protectant (such as maltodextrin, trehalose, glucose or skim milk). Lyophilization was conducted by quick freezing the mixture for 24 hours at −40° F., then placing into a lyophilizer for 3-4 days, using a shelf temperature of 86° F., a cold trap condenser below −94° F. and a vacuum below 15 mTorr. The dried microbes were homogenously pulverized using 100 μl strainer. The primary powder of the microbes was then vacuum packed and stored at 39.2° F.

The primary mixture of the above microbes may be added to the device with a suitable portion (e.g. about 40-70%) of a filler material. The filler material may be, for example, wooden chips such as cedar tree chips or oak tree chips. The filler material increases the efficiency and the rate of fermentation of the microbes by maximizing its contact area with food waste. The number of microbes is maintained at approximately 10⁶⁻⁹ cfu/g.

### Example 2

### Mixing with Filler Material and Production of Final Product

The primary powder of the microbe mixture (made in Example 1) was mixed with husks (the filler material) and protectant in the following ratio: 0.03527 ounces primary powder (approximately 1 gram), 0.67 ounces protectant, 2.2 pounds husks. This proportion was prepared for a 250 pound (or approximately 110 kilograms) daily capacity of food waste. The primary powder/filler material/protectant mixture was placed into a mixer and mixed for 30 to 60 minutes. The final product was packed and stored in cool dark place at room temperature.

### Example 3

### Breakdown of Food Waste

A food waste treatment device was prepared using the microbial mixture of Example 2. 40% of the volume of the container of the device was filled with the husks of Example 2. The husks were wet with enough water to inoculate the microorganisms. After 3-4 minutes, food waste was placed in the device. At this time, the microorganisms were activated and started to break down the waste, then discharged the water through the filter screen.

### Example 4

### Preparation of Bacterial Composition

Spray dried cultures of bacteria in power form are prepared, which contains the following four bacterial species: Bacillus amyloliguefaciens (ATCC No. 23842), Bacillus mojavensis (ATCC No. 51516), Bacillus pumilus (ATCC No. 14884) and Pseudomonas fluorescens (ATCC No. 13525). The bacterial concentration of the final product is about 2×10¹⁰ CFU/gram, with each of the four species constituting about 25% of CFUs.

Glycerine solution (IL) is prepared by mixing 750 ml of tap water and 250 ml of glycerine. 140 ml of the glycerine solution is sprayed onto 1.0 kg of corn cob fragments (Corn Cob Fractions, 5-8, obtained from Best Cob, LLC, 189 S. Bland Blvd., Independence Iowa) while mixing. The dampened corn cob fragments are allowed to dry overnight, and then the spray dried cultures of bacteria in power form are added to the fragments while mixing, allowing substantially all of the powder to adhere to the corn cob fragments, to produce the final product.

### Example 5

### Preparation of BIO-HELPER

In one embodiment of the invention, the filler material comprises a porous structure of polypropylene, also referred to as “BIO-HELPER” This section provides an example of how such polypropylene filler material may be prepared.

A base formula is prepared by mixing polypropylene with additives and water as per the mixture ratio (Polypropylene: 88.80%; Foaming Agent: 5.00%; Absorbent: 3.00%; H₂O: 0.20%; and Silicon: 3.00%). The mixed base formula is liquefied in an airtight chamber. The base formula turns into a fluid substance with high viscosity, which is extruded through a mold. The extruded substance is cooled down and cut into 2 mm wide and 2 mm long. The product has very rough surface by talc effect from liquation process and blowholes will be formed inside of material by foaming agent. And open cell will is formed by evaporation effect of inner gas from the material, when the material is exposed to air by being extruded from mold.

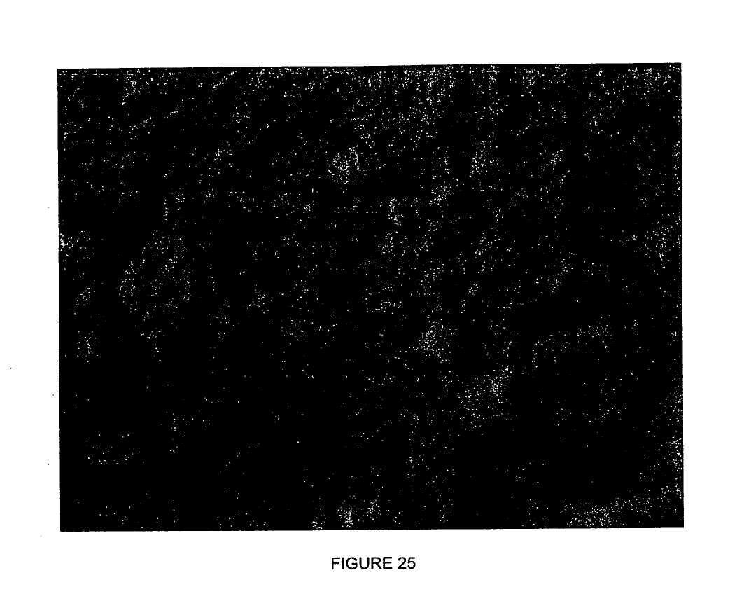

BIO-HELPER (the porous polypropylene filler material) has a large surface area and highly porous structure, which allows the surface and the holes to absorb microbes and moisture. FIGS. 24 and 25 depict photographs of BIO-HELPER, which illustrate the poriferous structure of Bio-Helper. Bio-Helper is specially designed and produced to have this poriferous structure.

### Bet Surface Area and Pore Analysis Report

A professional terminology “BET Surface Area” is often used to express the grade of porosity. This number represents the actual ratio of surface area for the material and bigger number signifies more pore in the material.

| TEST ITEM | TEST RESULT | TEST METHOD |
|---|---|---|
| BET Surface Area | 0.07 | KS L ISO 18757 |
| Gravity | 0.98 | KS M 0004 |
| Tensile Strength (length) | 39.7 N/m² | KS M 3006:2003 |
| Flexural Strength (length) | 37.5 N/mm² | KS M ISO 178:2002 |
| Flexural Mudulus {length) | 1.16 * 10³ | KS M ISO 178:2002 |

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1-75. (canceled)

76. A food waste treatment device comprising:
a food waste processing container configured to receive food waste for mixing with a microbial mixture via a container inlet, the food waste processing container housing a stirrer, the stirrer rotatably coupled to a driving motor to rotate in the food waste processing container, the food waste processing container including a discharge container configured to receive processed food waste and drain water out of the food waste processing container, the food waste processing container including a filter screen positioned between the stirrer and the discharge container; and
a food waste processing controller coupled to the food waste processing container, the food waste processing controller configured to control rotation of the stirrer.

77. The food waste treatment device of claim 76, wherein the microbial mixture comprises an effective combination of at least two species of microorganisms chosen from bacillus, lactobacillus, burkholderia, yeast fungus, eumycetes or any combinations thereof.

78. The food waste treatment device of claim 76, wherein the food waste processing container includes temperature sensor communicably coupled to the food waste processing controller and a heating element, the food waste processing controller configured to operate the heating element to regulate a temperature in the food waste processing container.

79. The food waste treatment device of claim 76, further comprising a humidity sensor communicably coupled to the food waste processing controller.

80. The food waste treatment device of claim 79, wherein the food waste processing container includes a water supply configured to supply water to the food waste processing container via a water inlet housed in the food waste processing container, the food waste processing controller configured to operate the water supply to control a humidity level in the food waste processing container, the water supply including a water injection nozzle upstream of the stirrer between the container inlet and the stirrer.

81. The food waste treatment device of claim 76, wherein the food waste processing container includes an oxygen supply, the food waste processing controller configured to operate the oxygen supply to regulate an oxygen level in the food waste processing container.

82. The food waste treatment device of claim 76, wherein the stirrer includes a stirrer axle extending along a rotation axis of the stirrer and a plurality of stirrer arms coupled to the stirrer axle and extending outward from the stirrer axle.

83. The food waste treatment device of claim 82, wherein a stirrer arm in the plurality of stirrer arms is coupled to the stirrer axle by a shear pin.

84. The food waste treatment device of claim 82, wherein a stirrer arm in the plurality of stirrer arms includes a blade having a polyurethane tip.

85. The food waste treatment device of claim 84, wherein the blades includes at least one of a spiral blade and a u-shaped stirrer arm.

86. The food waste treatment device of claim 76, wherein the food waste processing container includes a chopper coupled to the container inlet, the chopper configured to chop the food waste before it is agitated by the stirrer.

87. The food waste treatment device of claim 86, further comprising a funnel shaped hopper coupled to the container inlet, the hopper coupling the container inlet to the chopper.

88. A method of operating a food waste treatment device comprising:
inserting a microbial mixture and food waste into a food waste processing container via an inlet in the food waste processing container, the food waste processing container housing a stirrer, the stirrer rotatably coupled to a driving motor, the food waste processing container including a discharge container to receive processed food waste and drain water out of the food waste processing container, the food waste processing container including a filter screen positioned between the stirrer and the discharge container; and
actuating the stirrer via a food waste processing controller coupled to the food waste processing container.

89. The method of claim 88, further comprising controlling a temperature in the food waste processing container via the food waste processing controller.

90. The method of claim 89, wherein controlling the temperature includes maintaining the temperature between 20° C. to 35° C.

91. The method of claim 88, further comprising controlling a humidity level in the food waste processing container via the food waste processing controller causing an injection of water into the food waste processing container from a water supply coupled to the food waste processing container.

92. The method of claim 91, wherein controlling the humidity includes maintaining the humidity level between 65% to 75% humidity.

93. The method of claim 91, further comprising actuating a chopper coupled to the container inlet, the chopper configured to chop the food waste before it agitated by the stirrer.

94. The method of claim 88, further comprising stopping actuation of the chopper in response to opening a door coupled to the container at the container inlet.

95. The method of claim 88, wherein the microbial mixture comprises an effective combination of at least two species of microorganisms chosen from bacillus, lactobacillus, burkholderia, yeast fungus, eumycetes or any combinations thereof.


SEQUENCE LISTING (8 sequences)

SEQ ID NO: 1 - DNA, 845 bp, Bacillus subtilis
gcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgc

SEQ ID NO: 2 - DNA, 846 bp, Bacillus subtilis
gtgcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccg

SEQ ID NO: 3 - DNA, 843 bp, Burkholderia sp.
ttacacatgcaagtcgaacggcagcacgggtgcttgcacctggtggcgagtggcgaacgggtgagtaatacatcggaacatgtcctgtagtgggggatagcccggcgaaagccggattaataccgcatacgatccacggatgaaagcgggggaccttcgggcctcgcgctatagggttggccgatggctgattagctagttggtggggtaaaggcctaccaaggcgacgatcagtagctggtctgagaggacgaccagccacactgggactgagacacggcccagactcctacgggaggcagcagtggggaattttggacaatgggcgaaagcctgatccagcaatgccgcgtgtgtgaagaaggccttcgggttgtaaagcacttttgtccggaaagaaatccttggctctaatacagtcgggggatgacggtaccggaagaataagcaccggctaactacgtgccagcagccgcggtaatacgtagggtgcgagcgttaatcggaattactgggcgtaaagcgtgcgcaggcggtctgttaagacagatgtgaaatccccgggctcaacctgggaactgcatttgtgactggcaggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgttggggattcatttccttagtaacgtagctaacgcgtgaagttgaccgcctggggagtacggt

SEQ ID NO: 4 - DNA, 845 bp, Bacillus subtilis
gtgcctaatacatgcaagtcgagcggacagatgggagcttgctccctgatgttagcggcggcgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccg

SEQ ID NO: 5 - DNA, 1380 bp, Bacillus subtilis
ggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagttagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcgggtgaatacgttcccgggccttg

SEQ ID NO: 6 - DNA, 1432 bp, Bacillus subtilis
  misc_feature (58)..(58): n is a, c, g, or t
attgtagaggcatgctatacgcaggattagttattttaagtcacctgcagagtttgantaggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaaaccgcgaggttaagccaatcccacaaatctgttctcagttcggatcgcagtctgcaactcgactgcgtgaagctggaatcgctagtaatcgcggatcagcatgccgcgggtgaatacgttccc

SEQ ID NO: 7 - DNA, 1343 bp, Lactobacillus sp.
caggacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcg

SEQ ID NO: 8 - DNA, 1389 bp, Bacillus subtilis
tttttatacagctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatgcttgtttgaaccgcatggttcaaacataaaaggtggcttcggctaccacttacagatggacccgcggcgcattagttagttggtgaggtaacggctcaccaaggcaacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcgggtgaatacgttcccgggccttg